(19) United States
(12) Patent Application Publication
(10) Pub. No.: US 2005/0256435 A1
(43) Pub. Date: Nov. 17, 2005

(54) Clinical wound manager and method

(76) Inventor: Cathy Ilyse Hess, Harrisburg, PA (US)

Correspondence Address: DUANE MORRIS LLP;IP DEPARTMENT, 30 SOUTH 17TH STREET, PHILADELPHIA, PA, 19103-4196, US

(21) Appl. No.: 11/187,143

(22) Filed: Jul. 22, 2005

Related U.S. Application Data
(63) Continuation of application No. 09/626,366, filed on Jul. 24, 2000.
(60) Provisional application No. 60/146,006, filed on Jul. 28, 1999.

(51) Int. Cl. A61F 5/00

(52) U.S. Cl. 602/2

(57) ABSTRACT

A method is provided for assessing and documenting wound and skin conditions, and includes an automatically triggered alerting mechanism that is activated when a treatment is initiated that deviates or is a variation from an expected or standard treatment, under the current circumstances, and given previously gathered wound and skin condition patient information, and a method for triggering that alerting mechanism. In one embodiment the method includes the steps of initial patient care data gathering, clinical pathway identification and implementation, variance assessment and triggering, and issuance of variance cautions that are trigger during variance assessment and triggering, and are related to skin and wound care decisions made by healthcare professionals involved in a patient's care.

This application claims priority from copending provisional patent application titled: Clinical Wound Manager Method, filed Jul. 28, 1999, by the present inventor, and accorded Application Ser. No. 60/146,006.

### FIELD OF THE INVENTION

The present invention generally relates to the treatment of skin and wound conditions, and more particularly to systems and methods for determining the condition and appropriate treatment for skin and wound conditions.

### BACKGROUND OF THE INVENTION

Preventing and treating medical conditions related to a patient's skin, or a wound on the patient's body, are important activities for healthcare professionals and healthcare institutions, when a patient is in their care. Every year vast numbers of preventable and treatable skin and wound conditions occur, which transcend all age groups, all patient care settings, and which are rising to epidemic proportions. In 1995, there were an estimated four million chronic wounds associated with patients under care. Of the four million wounds, about two and a half to three million were attributed to diabetic and pressure ulcers leaving one to one and a half million attributable to venous leg ulcers. The costs associated with the treatment of non-healing wounds, and in particular chronic non-healing wounds is enormous, especially when the typical populations at risk (e.g., diabetic, paraplegic, or those otherwise suffering from neural and/or vascular impairment) are considered.

For example, the Clinical Trial Design Issues for Chronic Cutaneous Ulcers, Dermatologic and Opthamimic Drugs Advisory Committee (CDER) Meeting Jul. 14 and 15, 1997, the General Scientific Discussion reported chronic ulcers as a major health problem. In the United States alone, it is estimated that yearly cost for chronic ulcer care exceeds $2 billion dollars. The Agency for Health Care Policy Research (AHCPR, now the Agency for Healthcare Research and Quality, AHRQ), reports wound care for pressure ulcers at $200 billion dollars per year (including hospitalization, durable medical goods, home health, nursing home care, physicians, and transportation). This does not include the costs associated with emotional distress, pain and suffering.

The prevalence of pressure ulcers alone is between 3% and 30% in both the hospital and long-term care settings. Wounds caused by chemical, thermal or electric burns account for 75,000 hospitalizations annually. By 2030, it is estimated that 148 million people will have chronic skin and wound conditions, with associated annual direct costs increasing to about $798 billion. It is also documented that 15% of those patients diagnosed with diabetes will develop chronic leg conditions.

Once developed, treatment of an adverse skin or wound condition, that progresses beyond the initial phases, becomes prolonged, costly, and in many cases, difficult. With regard to decubitus ulcers (e.g., pressure ulcers, pressure sores, and bed sores), when an area of the body (e.g., a bony prominence) is subjected to an external surface for an extended period of time, the skin begins to breakdown. This breakdown is caused by normal blood flow becoming compromised due to pressure intensity. The greater the pressure, the shorter the time before intervention must be initiated, if negative effects are to be reversed. Normally, a person is capable of moving his or her body subconsciously and involuntarily, which tends to remove pressure from areas subjected to continuous pressure, shifting or redistributing body weight accordingly. By frequency of movement, tissue damage is avoided. When pressure is redistributed, a hyperemic action occurs, the body, under normal circumstances, automatically responds by allowing oxygen and metabolite restoration through increased blood supply and flow, with the tissue being restored to its normal state.

An aging population and increasing life expectancy is leading to an increase in the general age of the U.S. population. This will no doubt place an ever-increasing population at risk for the development of skin and wound medical conditions, particularly decubitus ulcers. Given the aging population, the subset of wounds and the cost of care associated with wounds, the clinical knowledge to care for this disease state is ongoing for all clinicians. With over 2000 wound care products retailed under 15 generic categories, clinicians need to be knowledgeable of a wound's etiology and the products to care for these wounds in order to treat effectively a patient's skin or wound condition.

Various methods are known for assessing skin and wound medical conditions, which include both subjective and objective assessments. There are also superficial assessment methods, which may be performed by a subjective or objective test (e.g., risk assessment, pain assessment, etc.). Some objective methods allow for quantitative assessment by measuring the size of the wound, photography, mapping, and volume measurement. Known approaches to wound assessment include both invasive and non-invasive techniques. Through lengthy clinical trials, extensive patient care, and new “high-tech” or “active” dressings, the number of effective treatments has increased, making it more difficult to determine best clinical therapies for a given skin or wound condition.

Traditionally, for health care providers who monitor patient care, a paper trail is left as supporting documentation of skin and wound condition assessment and treatment. This frequently results in poor information management including poor communication, intervention, and scheduling. This leads to patients not being treated promptly and properly, or fairly, which can lead to death and/or costly litigation.

Current information collection systems can often limit the number of patients that can be handled simultaneously by a given number of personnel. The management of this information can be burdensome, simply because of volume. This can lead to distractions, time lapse, decreased quality care, and other insufficiencies, including increased costs.

Conventional skin and wound condition information collection can lead to errors due to manual entry into a patient's file for each documented intervention. Such methods do not prompt other staff members to follow-up from one patient to the next, or from one phase of intervention to the next. This often leads to a more expensive and delayed response. Conventional methods also often make it difficult to access the information on a patient in a timely manner when, for example, a phone call requesting information regarding a patient is made by a health care professional.

Therefore, there is a need to automatically gather, track, benchmark, and organize the various aspects of a patient's skin and wound care needs.

Additionally, with all the demands being placed upon healthcare professionals, including case managers and clinical counterparts, and the population subjected to such risks, skin and wound care has not been high on the radar screen. While best efforts are provided, the knowledge and expertise necessary to deliver appropriate care is lacking. Professionals do not always have the tools necessary to note when an adverse effect is taking place before it is too late, or at least when proper intervention could have reversed an otherwise adverse effect.

There has therefore also been found to be a need to provide a system to assist care givers by detecting and alerting that a variance has occurred within the treatment process. A system is needed that will handle large numbers of patients and data, and support staff by promoting action and consistency with personnel for fewer deficiencies, and would be able to act like a “sub-case manager” and be applicable to any care setting.

### SUMMARY OF THE INVENTION

In a preferred embodiment, the present invention provides a process for assessing and documenting wound and skin conditions, and includes an automatically triggered alerting mechanism that is activated when a treatment is initiated that deviates or is a variation from an expected or standard treatment, under the current circumstances, and given previously gathered wound and skin condition patient information, and a method for triggering that alerting mechanism. In one embodiment the process includes the steps of initial patient care data gathering, clinical pathway identification and implementation, variance assessment and triggering, and issuance of variance cautions that are trigger during variance assessment and triggering, and are related to skin and wound care decisions made by healthcare professionals involved in a patient's care.

Initial patient care data gathering etiology, includes the gathering of objective and subjective data by a healthcare professional from both a patient and a treating clinician. The objective and subjective data may include a detailed history of past and present illnesses, a physical and thorough wound and skin assessment of the patient's entire body, a review of past and current medications and wound and skin care products, diet, activity level, elimination and hygiene needs, discharge planning, past and present consults, as well as other pertinent information and physical assessments. This gathered data is stored in a data base where it is searchable and retrievable.

Triggering mechanisms in the form of software programs that search and evaluate the data bases alert the clinician of a variance identified in the system based upon a variance detected between the prescribed treatment and the expected, most likely treatment identified from an appropriate clinical pathway associated with the particular diagnosed malady. The process and method of the present invention is most often implemented as software adapted to analyze the gathered data and provide a trigger mechanism for alerting the clinician when a variance is detected. The system does not dictate the clinical treatment pathway a clinician should employ. Rather, the triggered cautions are simply “gentle reminders” to the clinician that a deviation in a well known clinical treatment pathway was detected. In the invention, variances refer to the outcome that deviated from the core clinical treatment pathway based on the information entered by a clinician during treatment of a patient. An outcome, as used throughout this disclosure is meant to be the result, consequence, effect, or conclusion that is realized based upon a set of circumstances preceding a certain event. An outcome can be positive or it can be negative.

### BRIEF DESCRIPTION OF THE DRAWINGS

These and other features and advantages of the present invention will be more fully disclosed in, or rendered obvious by, the following detailed description of the preferred embodiment of the invention, which is to be considered together with the accompanying drawings wherein like numbers refer to like parts and further wherein:

FIG. 1 is a schematic block diagram of a general algorithm for use in developing each wound and skin condition type according to the invention

FIG. 2 is a schematic block diagram of an algorithm used to document aspects of a patient's health status according to the invention; and

FIG. 3 is a schematic block diagram of an algorithm for collecting wound/skin assessment data and treatment options according to the invention.

### DETAILED DESCRIPTION OF THE PREFERRED EMBODIMENT

This description of preferred embodiments is intended to be read in connection with the accompanying drawings, which are to be considered part of the entire written description of this invention. The present invention provides a method for assessing and documenting wound and skin conditions, and includes an automatically triggered alerting mechanism that advises a clinical healthcare provider or clinician, when he or she initiates a treatment that deviates or is a variation from an expected or standard treatment under the current circumstances or fails to initiate an expected or standard treatment. The method of the present invention may be practiced manually with the aid of physical documentation aids, e.g., a series of color coded forms or data entry and recording means, or may be implemented as software on a general purpose computer of the type well known in the art, either locally or as a part of a larger computer network, such as the Internet World Wide Web. Although in no way limiting, such a general purpose computer is preferably a general purpose Intel Pentium based personal computer, or equivalent, running a multi-tasking operating system, such as UNIX, LINUX, Microsoft Windows 95, 98 or NT. It will be understood that such a computer must include sufficient memory capacity to store a plurality of data associated with and representative of the clinical status of a patient's wound and skin according to the following techniques. A conventional display of the type normally used with such general purpose computers will often be used in connection with the practice of the present invention.

A plurality of pathways and algorithms (Tables 1 and 2) are known that define the step-by-step method associated with the treatment of a properly diagnosed malady of the skin and/or body surface. The steps associated with each pathway and algorithm are often quite specific to a particular diagnosis. A deviation from these known pathways often leads to a deterioration of the patient's condition. These pathways and algorithms are designed to be followed by the healthcare professionals that are assigned to care for the patient.

TABLE 1
PATHWAYS

Arterial Insufficiency Clinical Pathway
Diabetic Grade 1 Clinical Pathway
Diabetic Grade 2 Clinical Pathway
Diabetic Grade 3 Clinical Pathway
Diabetic Grade 4 Clinical Pathway
Diabetic Ischemic Clinical Pathway
Diabetic Neuropathic Clinical Pathway
Diabetic Non-Ischemic Clinical Pathway
Full Thickness Wound Clinical Pathway
Partial Thickness Wound Clinical Pathway
Prevention/At Risk Clinical Pathway
Stage 1 Pressure Ulcer Clinical Pathway
Stage 2 Pressure Ulcer Clinical Pathway
Stage 3 Pressure Ulcer Clinical Pathway
Stage 4 Pressure Ulcer Clinical Pathway
Venous Insufficiency Clinical Pathway

TABLE 2
ALGORITHMS

Pre-Admission Algorithm
Patient Admission and Clinical Implementation Algorithm
Wound Assessment Algorithm: A
Clinical Intervention Algorithm: B
Arterial Insufficiency: V9
Diabetic Ulcer Grade 1 Algorithm: D1
Diabetic Ulcer Grade 2 Algorithm: D2
Diabetic Ulcer Grade 3 Algorithm: D3
Diabetic Ulcer Grade 4 Algorithm: D4
Diabetic Ulcer Ischemic Algorithm: D5
Diabetic Ulcer Neuropathic Algorithm: D7
Diabetic Ulcer Non-Ischemic Algorithm: D6
Full Thickness Wound Algorithm: FTW
Infection / Managing Bacterial Colonization: I
Nutritional Assessment and Support: N
Partial Thickness Wound Algorithm: PTW
Post Surgical Assessment Algorithm: R
Pressure Ulcer Stage 1 Algorithm: P1
Pressure Ulcer Stage 2 Algorithm: P2
Pressure Ulcer Stage 3 Algorithm: P3
Pressure Ulcer Stage 4 Algorithm: P4
Prevention Algorithm: P
Support Surface Algorithm: L
Surgical Candidate Algorithm: S
Ulcer Care Algorithm: U
Vascular Evaluation Algorithm: VAS
Venous Insufficiency Algorithm: V10
Wound Management Algorithm: T

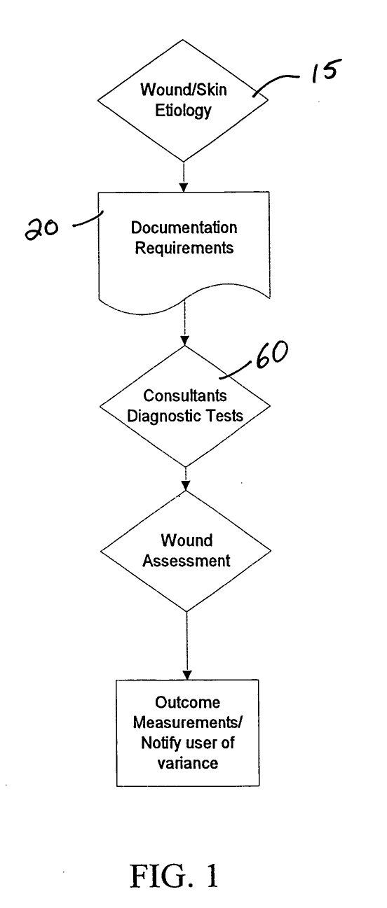

Referring to FIG. 1, the method of the present invention begins with the selection of a proper diagnosis of the malady afflicting the patient. More particularly, wound/skin etiology 15 is a method step, often implemented via a specialized screen portion of the display of the general purpose computer, that prompts the clinician to choose a diagnosis for the patient with skin and wound concerns. These skin and wound diagnoses are derived from the plurality of possible skin and wound diagnoses that would logically lead to one or more of the foregoing plurality of pathways and algorithms.

The health care professional/clinician must evaluate each patient's wound or skin condition by identifying the wound's cause, any underlying conditions and the wound's history to date. Establishing a cause for the wound or skin condition assists the clinician in identifying the correct classification and management approach to be used for selecting an appropriate pathway. For example, a wound caused primarily by pressure would be classified as a pressure ulcer, documented according to a known pressure ulcer staging system, and treated according to a specific pathway. Other wounds which may be vascular in nature, and result directly from arterial or venous insufficiencies, may be classified by depth and documented as, e.g., a vascular ulcer, partial, or full thickness. It will be understood that underlying medical conditions may be the cause of impaired wound healing and would need to be treated concurrently.

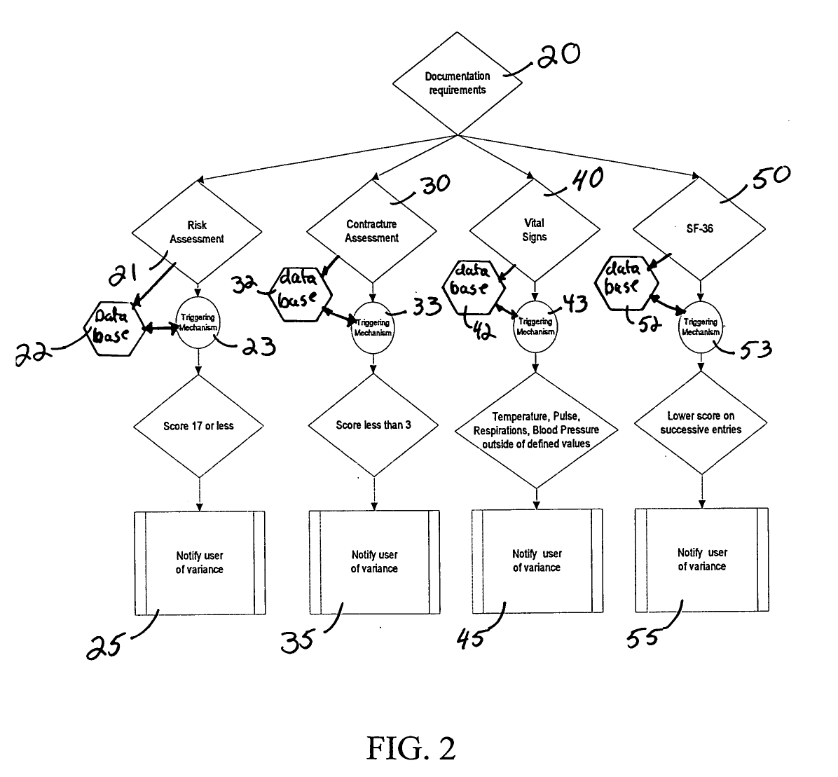

Documentation requirements 20 are fulfilled after the clinician has identified the correct diagnosis for the skin/wound care patient. In this step, a series of documentation requirements 20 must be completed (see FIG. 2) and the data stored in data storage means of the type well known in the art for use in connection with a general purpose computer.

For example, a risk assessment tool 21 for pressure ulcers may be applied to the patient. Here the well known Braden Scale may be employed to objectively characterize the condition of the skin and wound. Of course, it will be understood that other risk assessment tools that are useful in objectifying subjective information may be used with the present invention with equal effect. Rating scales are the most common risk assessment tools used by clinicians to identify the factors most closely associated with pressure ulcer formation and the potential risk in each patient. The commonly identified parameters of this type of scale are: sensory perception—defined as the ability to respond appropriately to pressure-related discomfort; moisture—defined as the degree to which skin is exposed to moisture; activity—defined as the degree of physical activity; mobility—defined as the ability to change and control body position; nutrition—defined as the usual food intake pattern; and friction and shear—described as the amount of force applied to the patient's body when the patient moves himself or herself or if a care giver slides in a bed or chair. Shear, which separates the skin from underlying tissues, and friction, which abrades the top layer of skin, also contribute to pressure ulcer development. Contributing systemic factors include infection, malnutrition, edema, obesity, emaciation, multi-system trauma, and certain circulatory and endocrine disorders. Each of the foregoing parameters are identified and assessed by the clinician, and a rating number is assigned to each parameter that corresponds to the clinician's objective assessment of the wound/skin condition. For example, a score of one to four is inherent in each parameter with a definition explaining the rationale for the score chosen. If the patient's combined score is, e.g., seventeen or less, the patient may be deemed at high risk for pressure ulcer development. It should be understood that the score of seventeen or less acts as a caution to the clinician that prescribed interventions for the patient are needed to decrease the risk of skin breakdown. These parameters, along with their assigned rating numbers, are stored at a known, searchable, and retrievable location in the memory of the general purpose computer. Thus a data base 22 of numbers that are associated with each of the parameters (e.g., Braden Scale) is created that is representative of the clinician's assessment of the patient's skin and wound condition.

According to the invention, the clinician's selection activity is monitored, via a variance trigger software program 23 that accesses data base 22, by reviewing each of the parameters, and identifying a most likely course of intervention to be followed, according to the pathways and algorithms, and based upon the objectified representation of the patient's skin/wound condition. Advantageously, if the clinician does not select protective interventions for the patient in accordance with the “most likely course of intervention”, a “caution” or alert code is triggered indicating that the clinician should screen for the proper support surfaces to decrease the likelihood of skin breakdown. In this way, the clinician has the opportunity to change his/her decision and to choose another product or proceed without altering the decision. A variance report 25 is then recorded and issued at the completion of each patient entry, based on the low score values identified.

In another aspect of the invention, a contracture assessment tool 30 is provided as a screening device that is used to identify areas of a patient's body that may be at risk for skin breakdown secondary to contracted body parts. A four part scoring system is typically used to identify the severity of the contracture by anatomical site. These sites include: ankle (left and right); elbow (left and right); fingers (left and right); hip (left and right); knee (left and right); shoulder (left and right); wrist (left and right). The four part scoring system defines the severity of the contracture by anatomical site. The scoring system is, e.g., one=Very limited range of motion; two=Moderate range of motion; three=Slightly limited range of motion; four=Full range of motion. These contracture parameters, along with their assigned rating numbers, are stored at a known, searchable, and retrievable location in the memory of the general purpose computer. Thus a data base 32 of numbers that are associated with each of the contracture parameters is created that is representative of the clinician's assessment of the patient's skin and wound condition related to contracture.

According to the invention, the clinician's selection activity is monitored, via variance trigger software program 33 that accesses data base 32, by reviewing each of the contracture parameters, and identifying a most likely course of intervention to be followed, according to the pathways and algorithms, and based upon the objectified representation of the patient's skin/wound condition as related to contracture. Should the patient score a one or two on any body part, the clinician would proceed to seek the appropriate interventions to prevent skin breakdown according to a selected one of the pathways. Advantageously, if proper interventions have not been identified in the system, a “caution” or alert code is triggered indicating that the clinician should reconsider the selected course of action. The clinician then has the opportunity to change his/her decision and choose another product or proceed without altering the decision. A variance report 35 is activated and recorded at the completion of each patient entry based on scoring values identified.

In yet another aspect of the invention, vital signs assessment 40 allows the clinician to enter the patient's vital signs, per visit. The vital signs reviewed with each patient entry include temperature, pulse, respiration, blood pressure, and weight. There are industry standard normal vital signs that are incorporated into the system. The clinician's selection of vital signs 40 is stored at a known, searchable, and retrievable data base 42 in the memory of the general purpose computer, and monitored (via variance trigger software program 43) by reviewing each of the vital signs recorded, and identifying a most likely course of intervention to be followed, according to the pathways and algorithms. Here again, if vital signs 40 entered by the clinician at each visit are out of range for the patient, a “caution” or alert code is triggered alerting the clinician of this/these findings. The clinician would then alert a physician of the abnormal findings. A variance report 45 is recorded and activated at the completion of each patient entry based on the abnormal vital sign values identified.

In yet another aspect of the invention, a short questionnaire (such as the SF-36 indicated generally by reference numeral 50) is often used to identify the patient's satisfaction with his/her health status. Of course, it will be understood that other patient self-evaluation and satisfaction questionnaires that are useful in gathering a person's own assessment of their health may be used with the present invention with equal effect. This questionnaire is administered to the patient typically prior to the first visit, during the middle of the patient's course of treatment and after the patient has received his/her full course of treatment, and asks the patient to quantify or score his/her perception of the status of their health. For example, a higher score would indicate the patient perceives his/her health as being better. These scores are recorded and stored in a searchable and retrievable data base 52 in the memory of the general purpose computer, so that if the score of successive questionnaires is lower than an initial questionnaire (as monitored by a variance trigger software program 53) a “caution” or alert code is triggered indicating that the clinician of this finding. A variance report 55 is recorded and activated at the completion of each patient entry based on the lower score identified on successive questionnaires.

Consultants/Diagnostic Tests 60 allows the clinician to enter the names of any consultant the patient may be seeing or any diagnostic test that may have been administered to the patient. This allows the clinician the opportunity to see these fields at-a-glance for reference points. If a consultant's name or diagnostic test is duplicated in the program (as monitored by a variance trigger software program) a “caution” or alert code is triggered alerting the clinician of these findings. A variance report is then recorded and activated at the completion of each patient entry based on the duplication of services entered.

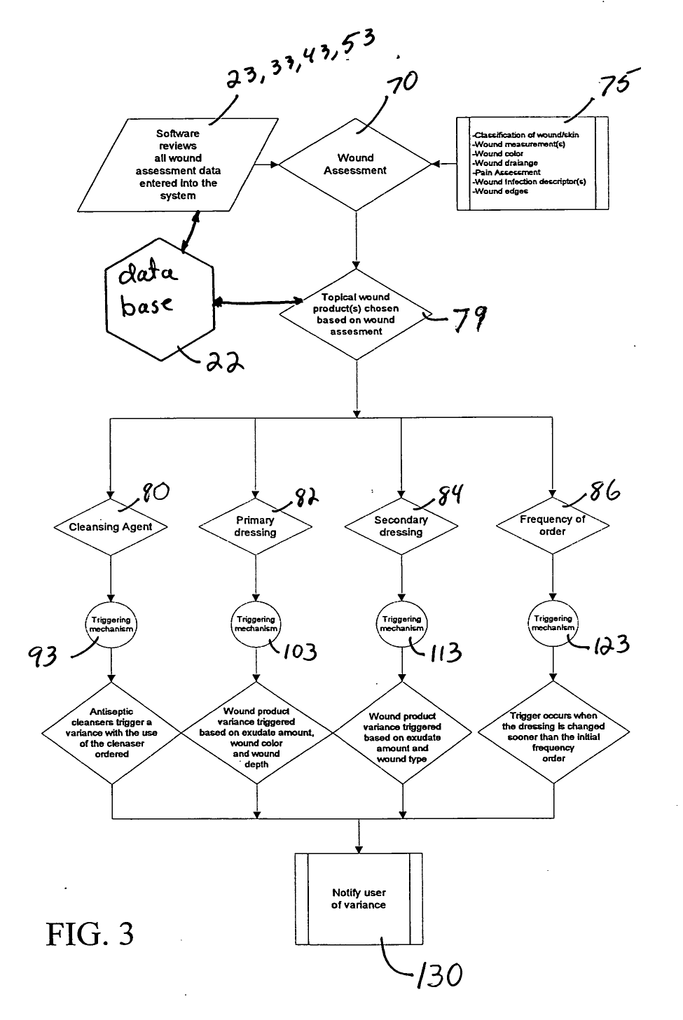

Turning to FIG. 3, the method of the invention begins with a detailed wound assessment designated 70. The purpose of wound assessment 70 is to collect the most complete data possible for identifying the cause of the altered skin. A data decision guide 75 aids the clinician by providing descriptors used-for collecting data. After accumulating the clinical data, the clinician enters the wound assessment findings into a memory location, e.g., data base 22, of the general purpose computer.

As an example of the pathway procedure that is monitored by the variance triggering software 23, 33, 43, and 53, based on the wound assessment information, the clinician must make a choice 79 the proper cleansing agent to clean the wound (generally referred to at 80), primary dressing to cover the wound (generally referred to at 82), secondary dressing to cover the primary dressing (generally referred to at 84), and document a frequency of order signifying how often the treatment is changed (generally referred to at 86). This step represents a complete wound care regimen that each clinician should obtain when treating a wound. Therefore, each wound assessed should have the foregoing regimen completed.

The goal of cleansing a wound is to remove bacteria and debris with as little chemical and mechanical trauma as possible and the same time, to protect healthy granulation tissue. A wound should be cleansed prior to each application of a primary dressing and a secondary dressing. The clinician is able to view different wound cleansers in the system when this field is opened. Choices for these cleansers include: acetic acid Betadine®); boric acid; Dakin's solutions; hydrogen peroxide; normal saline; wound cleanser, and/or bottle wound cleanser spray.

The clinician must choose a cleansing agent from the detailed list. If the clinician chooses an antiseptic cleanser such as Acetic Acid, Betadine®, Boric Acid, Dakin's solutions, hydrogen peroxide, triggering software 93 will recognize the clinician's selection, and issue a “caution” to the clinician regarding the cleanser of choice. The rationale behind this “caution” is the potential tissue damage and delay of wound healing that the antiseptic cleansers cause. Whereas, choosing other options, such as normal saline or bottle or spray wound cleanser, will allow the clinician to proceed through the system without any “caution”. The clinical rationales behind this are that these products provide a moist environment, promote granulation tissue formation and causes minimal fluid shifts in healthy cells. The clinician has the opportunity to change his/her decision and choose another product or proceed without altering the decision.

In another example, primary dressing 82 for the wound is defined as a dressing that is in physical contact with the wound bed. The clinician is able to view different primary dressings in the system when this field is opened. Choices for these primary dressings include alginates, biosynthetics, collagens, composites, compression therapy, contact layers, dermal skin replacements, enzymatic debriding agents, foams, gauzes, growth factors, hydrocolloids, hydrogels, specialty absorptives, transparent films, and wound fillers.

Inherent to each product listed are actions, indications, and contraindications. Each action, indication, and contraindication is built into the foregoing data bases, and utilized as a reference for triggering software 103, the system is able to detect if the wound information collected is compatible with the product chosen. If a variance is detected in the information entered, triggering software 103 issues a “caution” to the clinician regarding the primary dressing chosen based on the wound assessment entered. For example, if the product is inappropriate for the wound type and/or condition, based on the exudate amount, wound color, and wound depth, the caution will be issued by the triggering software. The clinician then has the opportunity to change his/her decision and choose another product or proceed without altering the decision.

In yet another example, secondary dressing for the wound 84 is a dressing that covers a primary dressing and serves to attach itself to the patient. The clinician is able to view different secondary dressings in the system, according to a properly identified pathway, when this field is opened. Choices for these secondary dressings include composites, compression therapy, foams, gauzes, hydrocolloids, hydrogels, specialty absorptives, and transparent films. Inherent to each product are actions, indications, and contraindications. Each action, indication, and contraindication is, and utilized as a reference for triggering software 113, so that the system is able to detect whether an appropriate secondary dressing is chosen for a wound, based on specific wound information. Thus the system is able to detect if the wound information collected is compatible with the product chosen. If a variance is detected in the information entered, triggering software 113 will issue a “caution” to the clinician regarding the secondary dressing chosen based on the wound assessment, i.e. the product is inappropriate based on the exudate amount, wound color, and wound depth. The clinician has the opportunity to change his/her decision and choose another product or proceed without altering the decision.

In a further example of the invention, how frequently the dressing is to be changed 86, as mandated in the physician's order, is monitored by the invention. More particularly, the clinician is able to view frequency 86 in the system when this field is opened. Choices for the frequency of the dressing change include Daily (QD), twice daily (BID), three times daily (TID), four times daily (QID), or as needed (PRN).

If the dressing is changed sooner than initially ordered, triggering software 123 will issue a “caution” to the clinician regarding the unplanned dressing change. If the dressing is changed sooner that the ordered length of time, the clinician has a responsibility to investigate the reason for the dressing change and attempt to correct the underlying reasoning or change the dressing to meet the patient's life-style.

A reporting mechanism 130 that takes place when a triggered variance event occurs at the completion of each patient entry. These reports detail the reason for the activation of the invention (trigger), the name of the field from which the variance is identified and the rationale for the variance.

It is to be understood that the present invention is by no means limited only to the particular constructions herein disclosed and shown in the drawings, but also comprises any modifications or equivalents within the scope of the claims.

1. A method for assessing deviations from a preselected medical treatment that has been indicated by appropriate diagnosis from a clinician, comprising the steps of:
(A) gathering patient care data and diagnosing a malady;
(B) storing said patient care data in a data storage means;
(C) identifying an appropriate clinical pathway to follow in treating said diagnosed malady;
(D) implementing said identified clinical pathway and recording clinical actions taken by a clinician in said data storage means;
(E) monitoring said recorded clinical actions taken by said clinician to determine variations from said identified clinical pathway; and
(F) alerting said clinician of a variance from said identified clinical pathway.

2. A method according to claim 1 wherein said gathering of said patient care data includes the use of a risk assessment tool comprising a rating scale to objectively characterize the condition of said patient's skin and wound.

3. A method according to claim 2 wherein said rating scales identifies factors most closely associated with the formation of a selected malady.

4. A method according to claim 3 wherein said factors are associated with parameters that are identified and assessed by said clinician, and a rating number assigned to each of said parameters that corresponds to said clinician's objective assessment of a wound/skin condition.

5. A method according to claim 4 wherein said a finite numerical score is selected from a preselected range and assigned to each of said parameters.

6. A-method according to claim 5 wherein a numerical score at or above a preselected value is indicative of a high risk for development of said malady.

7. A method according to claim 3 wherein said parameters, along with their assigned scores, are stored at a known, searchable, and retrievable location in said data storage means.

8. A method according to claim 7 wherein said monitoring includes reviewing each of said parameters, and identifying a most likely course of intervention to be followed by said clinician.

9. A method for assessing deviations from a preselected medical treatment that has been indicated by appropriate diagnosis from a clinician, comprising the steps of:
(A) gathering patient care data and diagnosing a malady;
(B) storing said patient care data in a data storage means of a general purpose computer;
(C) identifying an appropriate clinical pathway to follow in treating said diagnosed malady;
(D) implementing said identified clinical pathway and recording clinical actions taken by a clinician in said data storage means;
(E) monitoring said recorded clinical actions taken by said clinician to determine variations from said identified clinical pathway; and
(F) alerting said clinician of a variance from said identified clinical pathway.

10. A method according to claim 9 wherein said gathering of said patient care data includes observing and recording a patient's vital signs.

11. A method according to claim 10 wherein said recorded vital signs are each compared to a preselected value for said vital sign and monitored for deviations that are indicative of a high risk for development of a skin malady.

12. A method according to claim 9 wherein said implementing said identified clinical pathway and recording clinical actions taken by said clinician includes implementing a skin and wound care regimen.

13. A method according to claim 12 wherein said skin and wound care regimen are monitored for deviations that are indicative of a high risk for deterioration of said skin and wound.

14. A method according to claim 12 wherein said regimen comprises selection and application of dressings to a wound.

15. A method for assessing deviations from a preselected medical treatment that has been indicated by appropriate diagnosis from a clinician, comprising the steps of:
(A) gathering patient care data according to a predetermined regimen for diagnosing a malady of the skin;
(B) storing said patient care data in a data storage means of a general purpose computer;
(C) identifying an appropriate clinical pathway from a plurality of pathways for treating said diagnosed malady;
(D) implementing said identified clinical pathway via clinical actions taken by a clinician;
(E) monitoring said clinical actions taken by said clinician to determine variations from said identified clinical pathway; and
(F) alerting said clinician of a variance from said identified clinical pathway.

16. A method according to claim 15 wherein said regimen comprises answering a questionnaire that quantifies a patient's satisfaction with his/her health status.
